(19) United States
(12) Patent Application Publication
(10) Pub. No.: US 2003/0152996 A1
(43) Pub. Date: Aug. 14, 2003

(54) Method for nucleotide sequencing

(75) Inventor: Eiichiro Kimura, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 10/383,556

(22) Filed: Mar. 10, 2003

Related U.S. Application Data
(63) Continuation of application No. PCT/JP01/07671, filed on Sep. 5, 2001.

(51) Int. Cl. C12Q 1/68; C12P 19/34

(52) U.S. Cl. 435/6; 435/91.2

(57) ABSTRACT

The present invention relates to a method for nucleotide sequencing in which a nucleotide sequence of a single nucleotide or plural nucleotides can be determined by assaying reactions in one reaction solution. The nucleotide sequence can be determined according to the present method by detecting a reaction product continuously generated in an extension reaction system following the extension reaction of polynucleotide chain and thereby determining the alignment in the sequentially extending polynucleotide.

### CROSS REFERENCE TO RELATED APPLICATIONS

The present application claims priority to PCT International Patent Application No. PCT/JP01/07671, filed on Sep. 5, 2001, and Japanese Patent Application No. JP 2000-272489, filed on Sep. 8, 2000, which are hereby incorporated by reference in their entirety.

### BACKGROUND OF THE INVENTION

1. Field of the Invention

The present invention relates to a method for nucleotide sequencing.

2. Discussion of the Background

Due to the recent improvement of the nucleotide sequencing technology, a new research realm has been developed focusing on genome analysis. This focus has given rise to an enormous volume of nucleotide sequence data, perhaps best recognized via the determination of the overall human nucleotide sequence. Stemming from the glut of nucleotide sequence data, tools (such as software) for the genetic analysis of such an enormous volume of genetic information have been developed. Accordingly, future research and analysis of this genetic information will have increased importance in the development of pharmaceutical products and the industrialization of gene therapy and gene diagnosis. In such circumstances, expectantly, the nucleotide sequencing technology will achieve a far greater efficiency at lower cost.

The methods currently enjoying the most widespread use for nucleotide sequencing adopt the fundamental principles and methods introduced by Sanger, F., Nicklen, S., and Coulson, A. R. [(1977) Proc. Natl. Acad. Sci. USA 74, pp. 5463-5467] and Maxam A. M. and Gilbert, W. [(1977) Proc. Natl. Acad. Sci. USA 74, pp. 560-564], with Sanger's dideoxy sequencing method being the most commonly employed. Even to date, the “Sanger” method is an excellent method for nucleotide sequencing based on single-stranded DNA template.

The “Sanger” method is described as follows by separating the reactions at individual steps. During the polymerase extension reaction with a DNA template of unknown sequence, so as to elongate enzymatically the chain with four nucleotides, namely adenine (A), cytosine (C), guanine (G) and thymine (T). In this extension reaction, a dideoxynucleotide corresponding to each of the four nucleotides is randomly inserted in the position of the corresponding deoxynucleotide. Then, the resulting composite mixture is subsequently separated by electrophoresis on polyacrylamide gel or by using a capillary column packed with a gel, and the nucleotide sequence is determined thereby.

However, during the “Sanger” method molecular fractionation after dideoxynucleotide incorporation is inevitable. The step complicates a series of nucleotide sequencing procedures and requires a large assay system.

To address this deficiency in the “Sanger” method, various modifications have been made including the modification of the method of electrophoresis and the four types of deoxyribonucleotides and dideoxyribonucleotides utilized, as well as the use of fluorescent substances for detection. The relevant references describing these modifications include: G. M. Church and S. Kieffer-Higgins, Science, 240, pp. 185-188 (1988), International Publication WO 93/02212, Venter, C. J. et al., T.I.B.S. 10, pp.8-11 (1992), Prober, J. M. et al., Science 238, pp. 336-341 (1987), and Mathies, R. A. and Huang, X. C., Nature 359, pp. 167-169 (1992).

International Publication WO 93/02212 describes a method for identifying a nucleotide existing at a specific position in a nucleic acid sequence via dideoxynucleotide incorporation. In view of the requirement of electrophoresis, however, the method is identical to the aforementioned method. According to the method of WO 93/02212, a point mutation can be detected, but continuous nucleotide sequencing is impossible. Further, according to this method a primer should be arranged adjacent to the nucleotide, so it is impossible to characterize complicated mutations such as small insertion or deletion.

Methods that do not require the use of gel electrophoresis have also been proposed and employed, for example, nucleotide sequencing by hybridization [Strezoska, Z. et al., (1991) Proc. Natl. Acad. Sci. USA 88, pp. 10089-10093] and tunnel effect microscopy [Driscoll, R. J. et al (1990) Nature 346, pp. 294-296]. Due to progress in the DNA chip technology, hybridization techniques have been developed and enjoy frequent use. In this method; however, DNA for use in the hybridization should be first prepared by a technique such as PCR. When the hybridization method is used an additional problem of mismatch hybridization to a similar sequence (not the intended sequence) is unavoidable. Moreover, the hybridization method is not appropriate for the determination of a novel sequence spanning plural nucleotides, although the method can detect point mutation. Still further, the designing and preparation of a specific labeled probe is very cost intensive, as well as the costly nature involved to produce and use dideoxynucleotides.

Therefore, it is desirable even in the nucleotide sequencing of a specific region to directly determine a continuous sequence, preferably without the use of a hybridization method. Further, it is desirable to utilize a procedure that can be carried out rapidly by a simpler method. Still further, compounds to be used for the reaction are preferably inexpensive and commonly used. Additionally, it is important to reduce the cost by simplifying the assay apparatus and the reaction.

In an effort to address these problems, methods that do not use electrophoresis or hybridization are now proposed. Such a method involves a progress DNA extension reaction in a step-wise manner to characterize a nucleotide incorporated thereby. However, all of these progress DNA extension reactions proceed in a one-nucleotide by one-nucleotide fashion. Therefore, a cycle should be repeated, which includes a step of terminating the extension reaction intentionally for the replacement or addition of the reaction solution and a step of detecting an inserted nucleotide. Thus, this process is substantially more complicated.

International Publication WO 91/06678 describes a DNA method that does not use any gel electrophoretic methods, as well as an apparatus for carrying out the method. The reaction requires 3′-blocked dTNP. Further, the method requires a process of separating the reaction solution from template DNA to separate the reaction solution in one reaction container from the template DNA during extension. In addition, this method requires determination of inserted nucleotide using the reaction solution fractionated in another reaction container. For continuous determination of such a sequence, the cycle must be repeated. Therefore, the method is complicated in a practical sense.

Furthermore, WO 94/00346 describes a nucleotide sequencing method that does not require electrophoresis. Even by this method, DNA extension reaction is essentially progressed in a step-wise manner. Additionally, the method inevitably involves a separation process of the reaction solution from template DNA, by procedures such as the immobilization of the template DNA.

Accordingly, there remains a critical need for direct assay reactions that occur continuously in a single reaction solution to enable the determination of DNA sequence in real time.

### SUMMARY OF THE INVENTION

It is considered that an ideal mode of continuous plural determination of nucleotide sequence is a method including directly assaying reactions continuously occurring in a single reaction solution to enable the determination of DNA sequence in real time. However, it has been difficult to detect DNA sequences (alignment), which is supposedly positional information in a certain sense, based on reactions occurring in a single reaction solution. Previously, electrophoretic separation and step-wise DNA extension methods have been considered as an essential process for avoiding the tough problems.

The invention overcomes the problems and provides a method capable of determining a nucleotide sequence of a single nucleotide or plural nucleotides by assaying reactions in a single reaction solution.

The present method comprises continuously generating a reaction product in a nucleotide extension reaction of a polynucleotide chain; detecting the reaction product; and determining the alignment in the sequentially extending polynucleotide.

This process may be achieved by employing a DNA polymerase or an RNA polymerase is employed to facilitate the extension reaction of a polynucleotide chain. Further, the reaction product may be a fluorescent substance. In a particularly preferred embodiment, the nucleotide used as a starting substrate of the extension reaction of polynucleotide chain is a fusion substance of a fluorescent substance and a nucleobase; and wherein the reaction product is a fluorescent substance released from the fusion substance. The inventive method may use the DNA polymerase or RNA polymerase to catalyze the release of the reaction product from the fusion substance. Moreover, the fusion substance may be a deoxyribonucleotide 5′ triphosphate ester or a ribonucleotide 5′ triphosphate ester. In a preferred embodiment, the excitation and emission profile of the fluorescent substance varies, depending on each nucleotide as the substrate of the extension reaction.

The present invention also provides a method for comparing the gene sequences of plural biological species or different individuals, comprising: determining the nucleotide sequences of two or more polynucleotide chains using the method described above; and comparing the nucleotide sequences to detect the differences between the nucleotide sequences.

Further, the present invention provides a method for detecting a gene causing the difference in biological species or individuals, comprising employing the method of comparing the gene sequences of plural biological species or different individuals.

Also in an embodiment of the present invention is a diagnostic method, comprising: determining the nucleotide sequence of a test subject; determining the nucleotide sequences of a polynucleotide chain from a patient in need thereof; and comparing the nucleotide sequences to detect the differences between the nucleotide sequences. In this embodiment, the test subject may have a nucleotide sequence encoding a gene causing a disease selected from the group consisting of obesity and diabetes mellitus.

The above objects highlight certain aspects of the invention. Additional objects, aspects and embodiments of the invention are found in the following detailed description of the invention.

### DETAILED DESCRIPTION OF THE INVENTION

Unless specifically defined, all technical and scientific terms used herein have the same meaning as commonly understood by a skilled artisan in biochemistry, cellular biology, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

An extension reaction of a polynucleotide chain allows the extension of a polynucleotide chain by the sequential bonding of one nucleotide complementary to a nucleotide in a template polynucleotide sequence, in a manner dependent on the polynucleotide sequence. In this connection, it is possible to synthetically prepare a polynucleotide via chemical synthesis or an enzymatic reaction. Preferably, the extension reaction is facilitated by using a DNA polymerase or an RNA polymerase. DNA polymerases and RNA polymerases have esterase activity, whereby the ester bond in 3′-esterified nucleotide is cleaved (I. Rasolonjatovo and S. R. Sarfati, Nucleosides & Nucleotides, 18, 1021-1022 (1999)).

When a fusion substance comprising a nucleotide bound with a fluorescent substance via ester bond is used as a substrate for the polynucleotide chain extension reaction, as described below, the ester bond is therefore cleaved following the extension reaction of the polynucleotide chain, so that the fluorescent substance is released and sequentially detected. Accordingly, it is possible to determine the sequence alignment in the sequentially extending polynucleotide.

The phrase “reaction product continuously generated in the extension reaction system following the extension reaction of the polynucleotide chain” means a product generated during sequential bonding of one complementary nucleotide for the extension of polynucleotide chain, in a manner dependent on a template polynucleotide sequence. The reaction product may be any product that is detectable, such as a chemical substance or a physical signal. The chemical substance may be an organic substance or an inorganic substance. In a preferred embodiment the chemical substance is a radioisotope.

The phrase “continuous generation in the extension reaction system” means continuous occurrence of the extension reaction of polynucleotide chain and the generation of the reaction product in one reaction system, but never includes step-wise extension reaction to alternately progress the detection and the extension reaction separately to detect the reaction product at each step. Additionally, the reaction product herein may refer to the extending polynucleotide chain per se or a by-product generated following the polynucleotide extension reaction. In view of ready detectability described below, preferably, the by-product generated following the polynucleotide extension reaction is the reaction product in accordance with the invention. A more preferable reaction product is a fluorescent substance. By allowing a fluorescent substance with a different excitation and emission profile to be generated from each of the four nucleotides to be incorporated via the extension reaction, the excitation and emission profile may be detected, to thereby detect the four fluorescent substances. By detecting the fluorescent substances, the individual nucleotides incorporated via the extension reaction can be detected.

More preferably, nucleotides as the substrates of the extension reaction of polynucleotide chain are fusion substances with fluorescent substances, while the reaction product generated by the extension reaction of the polynucleotide chain is one of the fluorescent substances released from the fusion substances. More specifically, the fusion substance is deoxyribonucleotide 5′-triphosphate (dNTP) ester or ribonucleotide 5′-triphosphate (NTP) ester. As described above, the DNA polymerase or RNA polymerase to be used for the extension reaction has esterase activity cleaving the ester bond in 3′-esterified nucleotide. When a fusion substance of a nucleotide bound to a fluorescent substance via ester bond is used as a substrate of the extension reaction of polynucleotide chain, therefore, the ester bond is cleaved, following the extension reaction of polynucleotide chain, to release the fluorescent substance. By detecting the fluorescent substance, sequentially, the alignment in the sequentially extending polynucleotide can be determined.

The phrase “detection of the reaction product” means the detection of the presence of the reaction product in the extension reaction system or a reaction system partially separated from the reaction system, by a given detection method. The detection method varies, depending on the type of the reaction product generated. When different reaction products individually correspond to nucleobases A, T, G and C emerge; they are individually detected to determine the nucleotide types. In this case, such reaction products may sometimes be accumulated in the reaction system. When this occurs, the reaction products can be detected, sequentially, by examining the difference of the amount of freshly generated reaction products from that of the already existing corresponding reaction products.

Additionally, a method with no occurrence of the accumulation of any reaction product to be detected in the reaction system is more preferable. When the reaction product is to be a fluorescent substance, for example, a fluorescent substance with a different excitation and emission profile is fused with one of the four nucleotides as the substrates of the extension reaction. By subsequently detecting the excitation and emission profile using a fluorescent analyzer, the fluorescent substance can be detected. The detection of the fluorescent substance enables the detection of each nucleotide incorporated via the extension reaction.

The phrase “determination of the alignment in the sequentially extending polynucleotide” means the determination of nucleotide alignment in an objective polynucleotide as a consequence of the detection of a reaction product generated via the extension reaction of polynucleotide in a manner dependent on the sequence of a polynucleotide chain by using the polynucleotide intended for the nucleotide sequencing as template. When a reaction product emitting fluorescence at a wavelength varying in a manner dependent on the type of the nucleotide for use in extension is used, the change of the fluorescent intensity at each wavelength over time is measured to determine the nucleotide alignment.

When this method is employed, plural reaction product molecules may be generated simultaneously, depending on the plural template molecules. With simultaneous progress of the extension reactions of the plural molecules, the reactions are preferably synchronized among the molecules. Even when complete synchronization is impossible, a suitable computation procedure enables the calculation of the intended sequences. The computation procedure can be done via the analysis of the detected signals of such reaction products. Specifically, the computation can be performed by averaging or inductive optimization for a speculative objective sequence.

The phrase “comparison in gene sequence of plural biological species or different individuals to detect the difference therein” means comparison of homologous genes among higher organisms and prokaryotic organisms to identify the characteristics of the sequences of the genes. In this connection, higher organisms include humans, mouse, Drosophila, and nematodes. For an identical species, the term also means comparison in the characteristics of the sequence of a specific gene among individuals to identify the difference.

The phrase “diagnosis of the physiological feature of a biological species or an individual” means the identification of the relationship between physiological properties of a biological species or an individual and the nucleotide sequence thereof, to determine the characteristic properties of the biological species or the individual on the basis of the gene sequences thereof for establishing a diagnosis. Included within the term “diagnosis” is a use for the diagnosis of human diseases. Specifically, nucleotide sequence may be used for the examination of a gene type readily causing obesity or diabetes mellitus or for the diagnosis as to whether or not a specific pharmaceutical agent is therapeutically effective (Higuchi et al., Phermacogenetics 8, 87-(1998)). These are under way of research works and development, mainly as single nucleotide polymorphism analysis.

The phrase “detection of gene causing the difference in biological species or individuals” means to determine single nucleotide polymorphism and the like. Importantly, in this instance, information about nucleotide sequence is immediately available at low cost. Additionally, a larger number of nucleotide sequence samples are more effective; therefore, more rapid analysis of nucleotide sequence information at lower cost is a very significant advantage.

A specific mode for carrying out the invention is now described step by step summary:

1. Using a labeled dNTP (LdNTP) where the 3′-hydroxyl group of the deoxynucleotide is bound to a fluorescent substance via ester bond, and

2. progressing DNA extension reaction with a DNA polymerase,

3. incorporating a LdNTP and subsequently releasing the fluorescent substance via the esterase activity of the DNA polymerase per se and additional extensions with the nucleotide LdNTP are progressed continuously in one reaction solution.

4. the fluorescent substances continuously released following the extension reaction are sequentially detected by capitalizing on the fact that the fluorescent substance can emit fluorescence only after the fluorescent substance is released to label individually A, G, T and C with fluorescent substances emitting different fluorescence, to obtain the nucleotide sequence information.

Each of the steps is now described in more detail.

First, LdNTP can be prepared by the method described in the report of I. Rasolonjatovo and R. S. Sarfati, et al. (Nucleosides & Nucleotides, 16, 1757-1760 (1997) and Nucleosides & Nucleotides, 17, 2021-2025 (1998)). However, the type of fluorescent substance is not limited to those described therein. Additionally, any substance detectable is satisfactory, which is not necessarily a fluorescent substance. As described below, the length of the fluorescent life of fluorescent substance can improve the efficiency of the nucleotide sequencing in accordance with the invention. Still further, the fluorescent substance is not necessarily bound via ester bond but is bound via any mode of bonding in such a fashion that the bond can be cleaved with DNA polymerase or RNA polymerase in a manner dependent on the bonding with template, to regenerate a hydroxyl group.

Any commercially available DNA polymerase and RNA polymerase may be used within the present invention, so long as it is adequately supports the extension reaction. The reaction solution is of a fundamental composition including a template DNA containing a region for nucleotide sequencing, a primer for the extension reaction, a DNA polymerase or RNA polymerase, and any necessary solution additives. Such additives may include salts (such as salts of Mg and the like) and buffers for the efficient reaction with LdNTP and the polymerase selected.

By increasing the rate of the extension reaction with DNA polymerase and the like, additionally, the rate of the nucleotide sequencing in accordance with the invention can be increased. By decreasing the rate of the extension, inversely, the sensitivity of the nucleotide sequencing can be increased. The modification of the extension rate is satisfactorily obtained by a method for modifying the DNA polymerase per se and the like into variants or a method for modifying the conditions of the extension reaction. With the modification of DNA polymerase per se and the like, modification of not only the reaction rate but also the optimal range of the reaction temperature is also effective. In case of intending the modification of the reaction conditions, the modification can be done by adjusting the concentrations of substances to be added to the reaction solution, such as LdNTP, primer, template DNA and DNA polymerase or by adjusting the reaction temperature or pH.

LdNTP is incorporated in a manner depending on the sequence of template DNA. LdNTP itself never emits fluorescence. However, fluorescence is emitted, when LdNTP is incorporated into the extending DNA in a manner dependent on the template and the ester bond is subsequently cleaved. By individually labeling A, G, T and C with fluorescent substances emitting different fluorescence types, then, a nucleotide type incorporated can be identified. The emergence of hydroxyl group at position 3′ through the cleavage of the ester bond induces the insertion of a second LdNTP in a manner dependent on the template DNA sequence.

For fluorescent detection, the relation between the detection sensitivity and the time of resolution is important. The DNA extension reaction is a very rapid reaction under some conditions, but the reaction rate can be adjusted as described above. Principally, for example, the LdNTP concentration adjusted to a low concentration correspondingly lowers the reaction rate. For rapid nucleotide sequencing, it is suggested that extension reaction at a faster rate is preferable. However, the extension reaction should be set at a rate less than the time resolution of a detector when it is used for detecting fluorescence. Nonetheless, the time resolution of fluorescent analysis is generally as high as about 0.1 to 1.0 millisecond.

Compared with the methods for extension reaction using gel or in a step-wise manner as described above, which require several seconds to several minutes on a single nucleotide basis, the time resolution is far greater. At a rate corresponding to the time resolution of a fluorescence analyzer, in other words, nucleotide sequence can be determined by a method permitting sequential fluorescence emission using LdNTP. The detection sensitivity of fluorescence is so high that even positional molecule can be detected. By increasing the amount of template DNA, further, the fluorescent intensity emitted can be increased. It is then noted that in case of the existence of plural template DNA molecules, the polymerase extension reactions cannot be absolutely synchronized, so that emitted fluorescence is detected in the disorderly overlapped state. Even in this case, however, the original sequence can be determined by the computation process of the resulting data, as long as the plural template DNA molecules exist in a certain number. Practically, the optimal number of the DNA molecules to be extended and the optimal extension rate thereof are satisfactorily determined in view of the relation with the detection sensitivity and the reaction rate.

The method should be modified in that because fluorescent substances released via the esterase activity of DNA polymerase and the like accumulate in the reaction system, the fluorescent substances are also to be detected in an overlapped state with a fluorescent substance released from subsequent extension reaction. Therefore, the background for fluorescent detection is increased as the extension reaction progresses. However, the problem can be overcome by deleting the background by the computation process of the counted value. For nucleotide sequencing over a long time-course, efficient processing of the background is effective. Further, the development of a process never permitting re-excitation of once emitted fluorescent substance, if it is possible, will be more satisfactory.

Accordingly, the present invention enables nucleotide sequencing without a fractionation procedure by electrophoresis or step-wise repetition procedure. By using the method, the time and cost required for nucleotide sequencing can be remarkably improved, leading to the highly efficient gene analysis. Consequently, pharmaceutical products can be developed efficiently, while the cost for gene diagnosis or gene therapy can be reduced.

By using the inventive method, the procedures for nucleotide sequencing can be performed more rapidly in a simpler manner. The applicability thereof in application fields is thereby greatly improved, leading to the enlargement of the use thereof. To date, the technology for the analysis of human single nucleotide polymorphism is being developed. However, even the technology cannot establish the comparison at the whole genome level. When the overall genome sequences of a great number of humans may possibly be determined over a short period of time, the comparison among individuals at the whole genome level will be possible, only when the processing speed of computer is improved. Further, the simplification of the procedure for the determination of gene sequence enables gene diagnosis in a simpler manner, leading to the spread of gene diagnosis on clinical site more deeply.

Numerous modifications and variations on the present invention are possible in light of the above teachings. It is, therefore, to be understood that within the scope of the accompanying claims, the invention may be practiced otherwise than as specifically described herein.

What we claim is:

1. A method for nucleotide sequencing, comprising:
continuously generating a reaction product in a nucleotide extension reaction of a polynucleotide chain;
detecting the reaction product; and
determining the alignment in the sequentially extending polynucleotide.

2. The method according to claim 1, wherein a DNA polymerase or an RNA polymerase is employed to facilitate the extension reaction of a polynucleotide chain.

3. The method according to claim 1, wherein the reaction product is a fluorescent substance.

4. The method according to claim 3, wherein a nucleotide as a starting substrate of the extension reaction of polynucleotide chain is a fusion substance of a fluorescent substance and a nucleobase; and wherein the reaction product is a fluorescent substance released from the fusion substance.

5. The method according to claim 4, wherein a DNA polymerase or an RNA polymerase catalyzes the release of the reaction product from the fusion substance.

6. The method according to clam 4, wherein the fusion substance is a deoxyribonucleotide 5′ triphosphate ester or a ribonucleotide 5′ triphosphate ester.

7. The method according to claim 3, wherein the excitation and emission profile of the fluorescent substance varies, depending on each nucleotide as the substrate of the extension reaction.

8. A method for comparing the gene sequences of plural biological species or different individuals, comprising:
determining the nucleotide sequences of two or more polynucleotide chains using the method according to claim 1; and
comparing the nucleotide sequences to detect the differences between the nucleotide sequences.

9. A method for detecting a gene causing the difference in biological species or individuals, comprising employing the method according to claim 8.

10. A diagnostic method, comprising:
determining the nucleotide sequence of a test subject using the method according to claim 1;
determining the nucleotide sequence of a polynucleotide chain from a patient in need thereof using the method according to claim 1; and
comparing the nucleotide sequences to detect the differences between the nucleotide sequences.

11. The method of claim 10, wherein said test subject has a nucleotide sequence encoding a gene causing a disease selected from the group consisting of obesity and diabetes mellitus.
